(19) United States
(12) Patent Application Publication
(10) Pub. No.: US 2023/0375495 A1
(43) Pub. Date: Nov. 23, 2023

(54) BIOSENSOR AND RELATED METHOD OF MANUFACTURE

(71) Applicant: Lifescan IP Holdings, LLC, Malvern, PA (US)

(72) Inventors: Stuart Phillips, Malvern, PA (US); Zuifang Liu, Malvern, PA (US); Shona McConachie, Malvern, PA (US); Karn Campbell, Malvern, PA (US)

(21) Appl. No.: 17/749,759

(22) Filed: May 20, 2022

(51) Int. Cl. G01N 27/327; C12Q 1/00

(52) U.S. Cl. CPC G01N 27/3271; C12Q 1/006

(57) ABSTRACT

A biosensor includes a first base member and a parallel second base member disposed in a parallel and spaced relationship. At least one electrically conductive layer is deposited onto a facing surface of each of the first and second base members at respective first and second conductive regions. The first conductive region includes at least one layer made from a first electrically conductive material and the second conductive region includes at least one layer made from a second electrically conductive material, which is different from the first electrically conductive material. The first electrically conductive material is a precious metal with the first conductive regions of the biosensor defining cofacial electrodes and in which the second electrically conductive layer(s) is configured to electrically connect the cofacial electrodes to a test meter.

### TECHNICAL FIELD

This application generally relates to the field of biosensors and more specifically to a test strip, as well as a related method for manufacturing a test strip having one or more spaced electrically conductive material layers. Cofacial electrodes locally formed at one end of the test strip are made from a precious metal with the remainder of the electrically conductive layer(s) of the test strip being made from another electrically conductive material that is configured to enable electrical connection between the cofacial electrodes and a test meter.

### BACKGROUND

Biosensors are known in the field of diabetes management, such as test strips, onto which a blood sample can be deposited. One end of the test strip is inserted into a test meter that applies at least one predetermined voltage to two or more electrodes formed on the test strip. The electrodes are made from a precious metal, such as gold or palladium, in addition to at least one reagent layer that is applied to one of the electrodes. Upon application of the at least one predetermined voltage, electrochemical reactions are created wherein the resulting current can be measured in order to provide a determination of the concentration of blood glucose in the blood sample.

The inclusion of precious metals significantly impacts cost in the manufacture of the above described test strips. Accordingly, there is a prevailing need in the field to reduce the amount of precious metals used in the manufacture of biosensors, such as those described above, but without impacting functionality or overall performance.

### BRIEF DESCRIPTION

Therefore and according to an aspect of the present invention, there is provided a biosensor comprising a first base member and a second base member, the first and second base members being made from an insulative material that face one another in a spaced and parallel relationship. At least one electrically conductive layer is deposited onto a facing surface of each of the first and second base members and more specifically at a first conductive region and a second conductive region adjacent the first conductive region. According to at least one version, the first conductive region includes a deposited layer of a first electrically conductive material and the second conductive region includes at least a second electrically conductive material different from the first electrically conductive material, the first electrically conductive material being a precious metal and wherein the first conductive regions form cofacial electrodes of the biosensor.

According to at least one embodiment, the precious metal disposed in the first conductive region of the biosensor can be at least one of gold and palladium. Alternatively, other precious metals from the group consisting of platinum, iridium, osmium, rhenium, ruthenium, and rhodium can be used to form the cofacial electrodes. The second conductive material can be made from any suitable electrically conductive material including, but not limited to silver, copper, nickel, chromium and/or alloys thereof wherein the first and second conductive regions are electrically coupled to one another. According to one version, the first conductive region is defined by a single deposited layer of the precious metal. In another version, the first conductive region is defined by a first deposited layer made from the second electrically conductive material and a second deposited layer made from the precious metal that is applied onto the first deposited layer.

Preferably, the precious metal layer overlaps with a portion of the non-precious layer. The biosensor can further include a spacer or spacer layer, made from plastic or other insulative material, which is introduced between the electrically conductive material layers. The spacer layer includes a gap or spacing serving as a functional area in which electrochemical reaction take place in the presence of blood or other bodily fluid introduced into the biosensor. The biosensor further includes contact pads defined in the second electrically conductive regions that are connectable to a test meter configured to deliver at least one test voltage to the electrodes of the biosensor.

The base members facing one another in the biosensor can be a substrate or a film made from an electrically inert material. The precious metal layer can be applied by any suitable deposition process including physical vapor deposition (PVD) or chemical vapor deposition (CVD) processes. According to at least one embodiment, the precious metal layer is deposited with the second conductive region being masked in relation to the first conductive region. The second conductive metal layer(s) can also be applied by means of any suitable deposition process.

According to another aspect, there is provided a method for manufacturing a biosensor, the method comprising:
providing a first base member and a second base member;
depositing at least one electrically conductive layer onto respective first and second conductive regions of the first and second base members, in which a first electrically conductive material is deposited on each first conductive region and a second conductive material different from the first conductive material is deposited on each second conductive region adjacent the first conductive region in which the first electrically conductive material is a precious metal; and
disposing the first conductive regions of the first and second base members in spaced relation with one another and in which the first conductive regions of the biosensor form a pair of cofacial electrodes.

In at least one version, the second electrically conductive material is applied to the entire surface of each base member and then the first conductive material is deposited onto the second conductive material, but only in the first conductive region of the biosensor.

In another version, the first electrically conductive material can be deposited on the first conductive regions of each base member and the second electrically conductive material can be deposited on the second conductive regions. Preferably, a seam or other overlap is provided between the precious and non-precious material layers in order maintain electrical connectivity between the defined electrodes and a test meter.

According to the present invention, non-precious metals can be incorporated in combination with precious metals into a biosensor and more specifically an electrochemically-based test strip having cofacial electrodes using a mask during the deposition process in order to apply a well-defined layer of precious metal to cover at least the working area (that is, the area of the test strip in which a heterogeneous electrochemical reaction takes place) of the test strip. Advantageously, this design allows for the separation of two functions of the test strip, namely, the transmission of an electrical signal which is performed by the non-precious metal or alloy and the surface chemistry reactions, which are performed on the precious metal portion of the substrate layers.

An advantage provided is that existing precious metal layers can be used in combination with non-precious metal materials for use within a test strip production line without impacting overall performance. Accordingly, significant cost savings can be realized in terms of manufacturing the biosensor.

Another advantage is that by using a precious metal stripe in combination with a non-precious metal or alloy layer, the chemistry of the test strip is unchanged in terms of interaction with the existing precious metal surface.

Considerations such as surface energy, reagent adhesion, surface oxidation and adsorption of contaminants to the electrode surface are all substantially unchanged from prior known test strip designs.

In addition, the control of non-precious metal or alloy layer thickness according to the presently described method allows for resistance values to be tailored to match those of the precious metals, thereby providing a conductive pathway or bridge for electrical signal transmission with a resistance equivalent to that of the precious metals (e.g., palladium and gold).

Furthermore, the use of non-precious metal or alloys, having a greater hardness than that of precious metals, particularly gold, improves robustness of the electrical contacts of the test strip with the connectors of the test meter.

These and other features and advantages will be readily apparent from the following Detailed Description, which should be read in conjunction with the accompanying drawings.

### BRIEF DESCRIPTION OF THE DRAWINGS

FIG. 1A is a top perspective view of a known test strip;

FIG. 1B is an exploded version of the top perspective view of the test strip of FIG. 1A;

FIG. 1C is an enlarged perspective view of a distal portion of the test strip of FIGS. 1A and 1B;

FIG. 1D is a side elevational view, taken in section, of the distal portion of the test strip of FIGS. 1A-1C;

FIG. 2 is a schematic view of a test meter coupled to the test strip of FIGS. 1A-1D;

FIG. 3 is a representative voltage waveform used in connection with the test meter of FIG. 2 for application to the test strip of FIGS. 1A-1D;

FIG. 4 is an exploded perspective view of a test strip made in accordance with aspects of the present invention;

FIG. 5 is a top perspective view of the test strip of FIG. 4;

FIG. 6(a) is a top view of a portion of a test strip made in accordance with aspects of the present invention;

FIG. 6(b) is a side elevational view of the portion of the test strip of FIG. 6(a);

FIG. 7 is a side elevational view of a test strip, shown in exploded form, and made in accordance with aspects of the present invention;

FIG. 8 is a side elevational view of a portion of the test strip of FIG. 7;

FIGS. 9(a) and 9(b) are respective top and side elevational views of the portion of the test strip of FIGS. 7 and 8, depicting an overlap region;

FIG. 10 depicts a side elevational view of another test strip made in accordance with aspects of the present invention; and

FIG. 11 depicts a side elevational view of another test strip made in accordance with aspects of the present invention.

### DETAILED DESCRIPTION

The following describes several embodiments of biosensors used for determining at least one analyte of interest (e.g., blood glucose), as well as a related method for manufacturing a biosensor. More specifically, each of the herein described biosensors and related manufacturing method(s) relate to a test strip having one or more facing (cofacial) electrodes defining an electrochemical cell in which electrical potentials are driven in the presence of an enzyme or other reagent applied to at least one of the electrodes to create electrochemical reactions in the presence of a bodily fluid sample (e.g., whole blood). It will be understood, however, that the presently disclosed invention can be used, in principle, with any type of electrochemical cell having spaced apart cofacial electrodes and a reagent layer. For example, an electrochemical cell can be in the form of a test strip. In one aspect, the test strip may include two opposing electrodes separated by a thin spacer layer, defining a sample-receiving chamber or zone in which a reagent layer is positioned on one of the opposing electrodes. One skilled in the art will appreciate that other types of test strips, including those with configurations having more than two facing electrodes, may also be used in accordance with the methods described herein.

In addition, a number of terms are used throughout the following description to provide a suitable frame of reference with regard to the accompanying drawings. These terms, which include “first”, “second”, “distal”, proximal”, “above”, “below”, “top”, “bottom” and the like are not intended to overly narrow the intended scope of the present invention, except where so specifically indicated. Additionally, the accompanying drawings are intended to adequately depict salient features of the present invention. Accordingly, the drawings are not necessarily to scale and should not be used for scalar purposes.

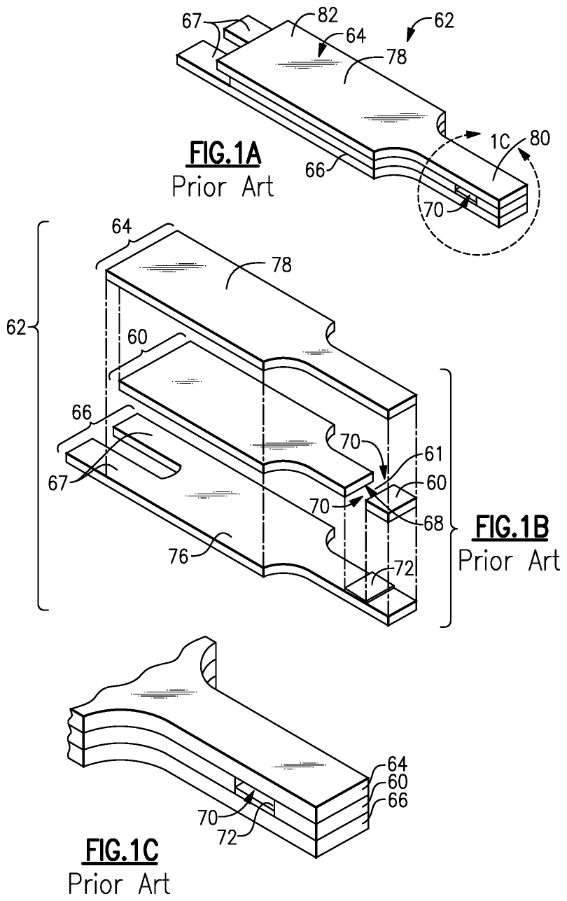

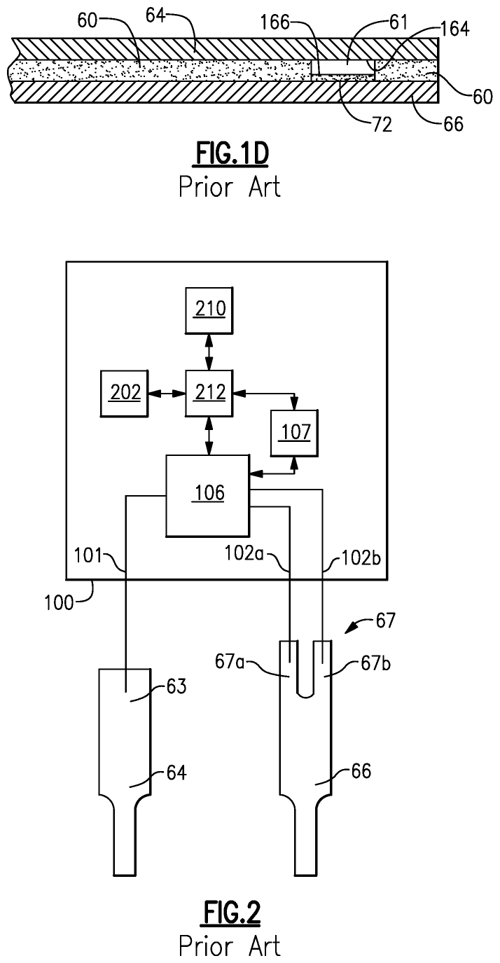

For purposes of background, FIGS. 1A-1D each depict a known biosensor in the form of a test strip 62 having a plurality of stacked layers in which the test strip 62 is further defined by a distal end 80 and an opposing proximal end 82. The distal end 80 of the test strip 62 includes a formed sample receiving chamber 61 and the proximal end 82 of the test strip 62 is configured for connection to a test meter, schematically shown in FIG. 2. More specifically, the test strip 62 includes a pair of base or substrate members 64, 66, each of which are made from a suitable plastic material. When assembled, each of the base members 64, 66 are defined by an interior or inner facing surface and an opposing exterior or outer facing surface. Each of the interior facing surfaces of the base members 64, 66 are entirely coated with metallized films. More specifically, the interior surface of the upper base member 64 is coated with gold forming a metallized gold layer and the interior surface of the lower base member 66 of the test strip 62 is coated with palladium, thereby forming a metallized palladium layer. Each of the precious metal layers cover the entire interior facing surface of the formed test strip 62 wherein the precious metal layers are applied to the base members 64, 66 using a sputtering process.

Sandwiched between the upper and lower precious metal layers is a spacer layer 60, which is also made from a suitable plastic. The spacer layer 60 includes a cut-out region 68 adjacent the distal end 80 of the test strip 62 that provides a predetermined spacing or gap between the metallized film layers and cooperates to form the sample receiving chamber 61. A reagent layer 72 which includes an enzyme/mediator is deposited onto a portion of the surface of the metallized palladium layer at the distal end 80 of the test strip 62, forming cofacial electrodes of the test strip 62 separated by the spacer layer 60 extending through the spacing 68. The test strip 62 further includes one or more electrode connection points or contact pads 67 formed at the proximal end 82 of the test strip 10 wherein the gold and palladium layers define electrodes in a working area of the test strip and electrode track connections to the contact pads 67, as shown in FIGS. 1A-1D.

The applied metallized film layers commonly provide upper and lower cofacial electrodes 166, 164, connection tracks 76, 78, and contact pads 67, 63, where the connection tracks 76, 78 of each metallized film layer electrically connects the electrodes 166, 164 to the contact pad 63, 67, as shown in FIG. 1B. The first electrode 166 is a portion of the applied metallized layer that is immediately beneath the reagent layer 72, shown in FIG. 1B. Similarly, the second electrode 164 is a portion of the remaining metallized film layer above the reagent layer 72.

The sample-receiving chamber 61 is defined by the first electrode 166, the second electrode 164, and the spaced gap or cutout area 68 formed in the spacer 60 near the distal end 80 of the test strip 62, as shown in FIGS. 1B and 1D. The first electrode 166 and the second electrode 164 define the bottom and the top of the sample-receiving chamber 61. The cutout area 68 of the spacer 60 defines the sidewalls of the sample-receiving chamber 61. The sample-receiving chamber 61 includes one or more ports 70 that provide a sample inlet and/or a vent. In this version depicted, two ports 70 are provided in which one of the ports, see arrows 70, allows a fluid sample to ingress and the other port 70 acts as a vent.

The sample-receiving chamber 61 of this described test strip 62 has a small volume in the range of from about 0.1 microliters to about 5 microliters, about 0.2 microliters to about 3 microliters, or, preferably, about 0.3 microliters to about 1 microliter. To provide the small sample volume, the cutout 154 has an area ranging from about 0.01 cm² to about 0.2 cm², about 0.02 cm² to about 0.15 cm², or, preferably, about 0.03 cm² to about 0.08 cm². In addition, the formed electrodes 166, 164 are spaced apart in the range of about 1 micron to about 500 microns, preferably between about 10 microns and about 400 microns, and more preferably between about 40 microns and about 200 microns. The relatively close spacing of the electrodes 166, 164 also allows redox cycling to occur, where oxidized mediator generated at the first electrode can diffuse to the second electrode to become reduced, and subsequently diffuse back to the first electrode to become oxidized again.

For the reagent layer 60, examples of suitable mediators include ferricyanide, ferrocene, ferrocene derivatives, osmium bipyridyl complexes, and quinone derivatives. Examples of suitable enzymes include glucose oxidase, glucose dehydrogenase (GDH) using a pyrroloquinoline quinone (PQQ) co-factor, GDH using a nicotinamide adenine dinucleotide (NAD) co-factor, and GDH using a flavin adenine dinucleotide (FAD) co-factor [E.C.1.1.99.10].

In terms of operability, either the gold layer or the palladium layer can perform the function of a working electrode of the test strip 62, depending on the magnitude and/or polarity of an applied test voltage from a test meter coupled to the test strip 62. The working electrode may measure a limiting test current that is proportional to the reduced mediator concentration. For example, if the current limiting species is a reduced mediator (e.g., ferrocyanide), then it can be oxidized at the first electrode 166 as long as the test voltage is sufficiently more positive than the redox mediator potential with respect to the second electrode 164. In such a situation, the first electrode 166 performs the function of the working electrode and the second electrode 164 performs the function of a counter/reference electrode.

Similarly, if the test voltage is sufficiently more negative than the redox mediator potential, then the reduced mediator can be oxidized at the second electrode 164 as a limiting current. In such a situation, the second electrode 164 performs the function of the working electrode and the first electrode 166 performs the function of the counter/reference electrode.

Initially, performing an analysis using the herein described test strip 62 includes introducing a quantity of a fluid sample into the sample-receiving chamber 61 via one of the ports 70. The port 70 and/or the sample-receiving chamber 61 are configured such that capillary action causes the fluid sample to fill the sample-receiving chamber 61. The first electrode 166 and/or second electrode 164 may be coated with a hydrophilic reagent to promote the capillarity of the sample-receiving chamber 61.

FIG. 2 provides a simplified schematic showing a test meter 100 interfacing with a first contact pad 67 and a second contact pad 63 of the test strip 62. The second contact pad 63 can be used to establish an electrical connection to the test meter 100 through a U-shaped notch 65, as illustrated in FIGS. 1A-1D. In one embodiment, the test meter 100 may include a second electrode connector 101, and first electrode connectors 102a, 102b, a test voltage unit 106, a current measurement unit 107, a processor 212, a memory unit 210, and a visual display 202, as schematically shown in FIG. 2. The first contact pad 67 includes two prongs 67a, 67b. The first electrode connectors 102a, 102b separately connect to the prongs 67a, 67b, respectively. The second electrode connector 101 can connect to the second contact pad 63. The test meter 100 is configured to measure the resistance or electrical continuity between the prongs 67a, 67b to determine whether the test strip 62 is electrically connected to the test meter 100. One skilled in the art will appreciate that the test meter 100 can use a variety of sensors and circuits to determine when the test strip 62 or variants thereof is properly positioned with respect to the test meter 100.

The test meter 100 is configured to apply a test voltage and/or a current between the first contact pad 67 and the second contact pad 63. Once the test meter 100 recognizes that the test strip 62 has been inserted, the test meter 100 turns on and initiates a fluid detection mode. In one embodiment, the fluid detection mode causes the test meter 100 to attempt to apply a voltage such that a constant current of about 0.5 microampere would flow between the first electrode 166 and the second electrode 164. Because the test strip 62 is initially dry, the test meter 100 measures a relatively large voltage, which can be limited by the maximum voltage that the test meter 100 is capable of supplying. When the fluid sample bridges the gap between the first electrode 166 and the second electrode 164 during the dosing process, the test meter 100 will measure a decrease in applied voltage and when it is below a predetermined threshold will cause the test meter 100 to automatically initiate a glucose test sequence.

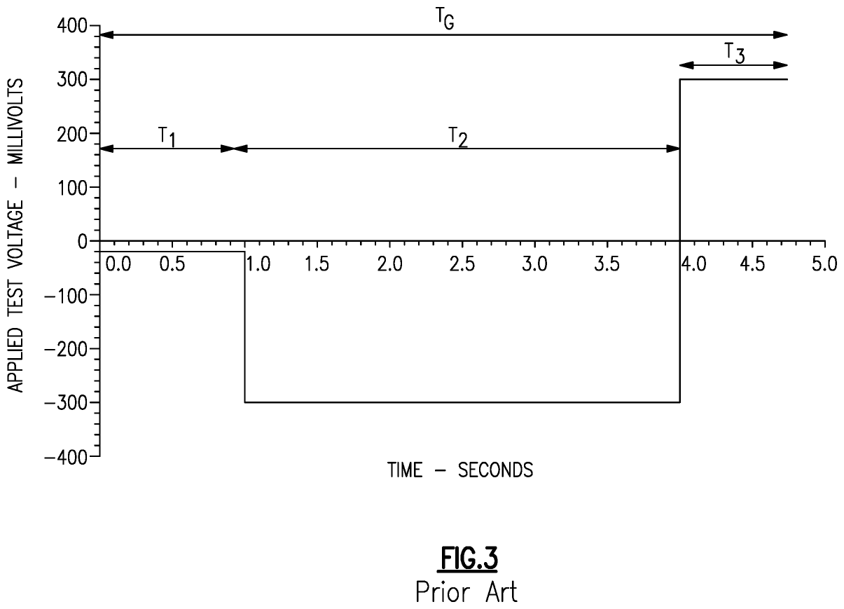

In the version herein described in this background, the test meter 100 can perform a glucose test by applying a plurality of test voltages for a number of prescribed intervals, as shown in FIG. 3. The plurality of test voltages may include a first test voltage V₁ applied for a first time interval T₁, a second test voltage V₂ applied for a second time interval T₂, and a third test voltage V₃ applied for a third time interval T₃. A glucose test time interval T_{G} represents an amount of time to perform the glucose test (but not necessarily all the calculations associated with the glucose test). The glucose test time interval T_{G} can range from about 1 second to about 15 seconds or longer and more preferably from about 1 second to about 5 seconds, as shown in FIG. 3. The plurality of test current values measured during the first, second, and third time intervals may be performed at a frequency ranging from about 1 measurement per nanosecond to about one measurement per 100 milliseconds. While an embodiment using three (3) test voltages in a serial manner is described, one skilled in the art will appreciate that a glucose test sequence may include different numbers of open-circuit and test voltages. For example, as an alternative embodiment, the glucose test sequence could include an open-circuit for a first time interval, a second test voltage for a second time interval, and a third test voltage for a third time interval. One skilled in the art will appreciate that names “first,” “second,” and “third” are chosen for convenience and do not necessarily reflect the order in which the test voltages are applied. For instance, an embodiment can have a potential waveform where the third test voltage can be applied before the application of the first and second test voltage.

Once the glucose assay has been initiated, the test meter 100 may apply a first test voltage V₁ (e.g., about −20 mV as shown in FIG. 3) for a first time interval T₁ (e.g., about 1 second). The first time interval T₁ can range from about 0.1 seconds to about 3 seconds and preferably range from about 0.2 seconds to about 2 seconds, and most preferably range from about 0.3 seconds to about 1 seconds.

The first time interval T₁ may be sufficiently long so that the sample-receiving chamber 61 can fully fill with sample and also so that the reagent layer 72 can at least partially dissolve or solvate. The reagent layer 160 has an area larger than the area of the formed electrode in which a portion of the spacer layer 150 can overlap and contact the reagent layer 160. In one aspect, the first test voltage V₁ may be a relatively low value so that a relatively small amount of a reduction or oxidation current is measured. Typically, a relatively small amount of current is observed during the first time interval T₁ compared to the second and third time intervals T₂ and T₃. For example, when using ferricyanide and/or ferrocyanide as the mediator, the first test voltage V₁ can range from about −100 mV to about −1 mV, preferably range from about −50 mV to about −5 mV, and most preferably range from about −30 mV to about −10 mV.

After applying the first test voltage V₁, the test meter 100 applies a second test voltage V₂ between the first electrode 166 and the second electrode 164 (e.g., about −0.3 Volts as shown in FIG. 3), for a second time interval T₂ (e.g., about 3 seconds as shown in FIG. 3). The second test voltage V₂ may be a value sufficiently negative of the mediator redox potential so that a limiting oxidation current is measured at the second electrode 64. For example, when using ferricyanide and/or ferrocyanide as the mediator, the second test voltage V₂ can range from about −600 mV to about zero mV, preferably range from about −600 mV to about −100 mV, and more preferably be about −300 mV.

The second time interval T₂ should be sufficiently long so that the rate of generation of reduced mediator (e.g., ferrocyanide) can be monitored based on the magnitude of a limiting oxidation current. Reduced mediator is generated by enzymatic reactions with the reagent layer 72. During the second time interval T₂, a limiting amount of reduced mediator is oxidized at the second electrode 164 and a non-limiting amount of oxidized mediator is reduced at the first electrode 166 to form a concentration gradient between the first electrode 166 and the second electrode 164. Additional details relating to the manufacture and testing of the test strip 62 are described in U.S. Pat. Nos. 8,529,751 and 8,449,740, the entire contents of each being herein incorporated by reference.

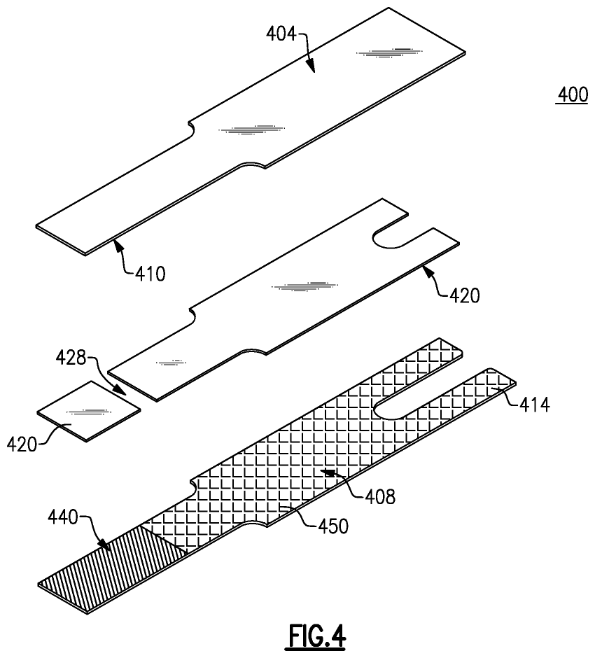

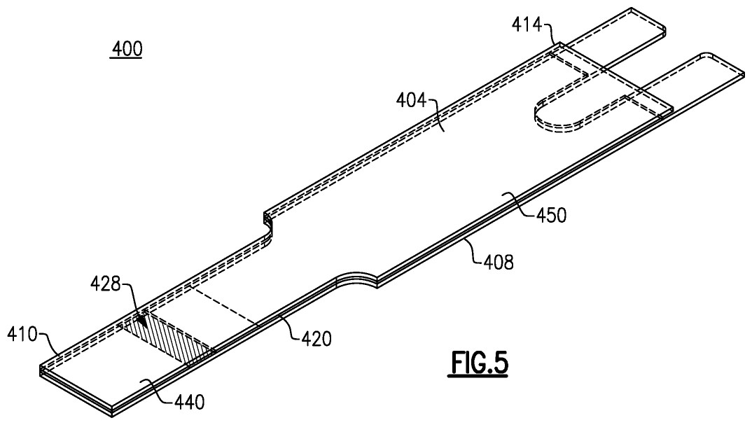

With the preceding background, FIGS. 4 and 5 depict the basic configuration of the test strip 400 made in accordance with the present invention. More specifically, the test strip 400 includes all of the structural components of the previously described test strip 62, FIGS. 1A-1D, and are configured for connection to a test meter, such as previously shown in FIG. 2 for applying a test waveform, such as shown in FIG. 3. More specifically, the test strips herein described in accordance with the present invention commonly include respective upper and lower base members 404, 408, as well as a spacer layer 420 sandwiched between the upper and lower base members 404, 408, the spacer layer 420 including a cut out area 428 adjacent a distal end 410 of the test strip 100. However and unlike prior known versions, precious metal layers are applied only to a local portion of one or both of the base members 404, 408, this local portion herein referred to as a first conductive region 440, which is disposed at the distal end 410 of the test strip 400 and which includes the cutout area 428 of the spacer layer 420. As previously discussed, the upper and lower base members 404, 408 are each made from a suitable insulative plastic material that can be provided as a rigid planar sheet, or alternatively in a film format.

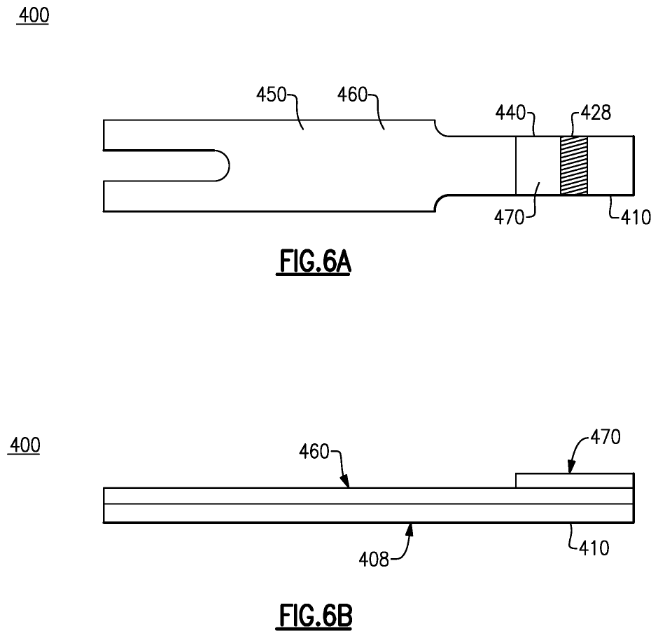

FIG. 4 shows an exploded version of the test strip 400 (the reagent layer not shown in this view for clarity) showing the first conductive region 440 and an adjacent second conductive region 450 that essentially is defined by the remainder of the facing surface of the upper and lower base members 404, 408, respectively. In FIG. 4, the first and second conductive regions 440, 450 of only the lower base member 408 are actually shown, but it should be understood that the corresponding features of the upper base member 404 are identical. The first conductive region 440 includes the cutout area 428 of the spacer layer 420, which also defines the working area shown by darkened area 428 of the test strip 400, best shown in FIG. 5. Specific embodiments relating to the localized deposition of the metallized precious metal layers are now described with reference to FIGS. 6(a)-11.

According to a first embodiment shown in FIGS. 6(a) and 6(b), a first electrically conductive material layer 460 is applied to the interior facing surfaces of the upper and lower base members 404, 408, respectively of a test strip 400. Only the lower base member 408 is shown. For purposes of the present invention, the first electrically conductive material (e.g., non-precious metal) can include copper, silver, nickel, chromium or alloys thereof (such as nichrome) may be used as the material for electrical signal transmission. Literally any material with an appropriate electrical conductivity may be used for the first electrically conductive layer 460. The first electrically conductive material layer 460 can be applied by any suitable deposition technique, which may include physical vapor deposition (PVD) or chemical vapor deposition (CVD) processes over the entire interior facing surface of each of the upper and lower base members 404, 408 according to this specific embodiment.

According to this first embodiment and adjacent the distal end 410 of the test strip 400, a second electrically conductive material layer 470 is deposited directly onto a portion of the first electrically conductive layer 460 of each of the upper and lower base members 404, 408. As shown in FIGS. 6(a) and 6(b), only the lower base member 408 is depicted but it will be understood the upper base member 404 is similarly processed. More specifically, a strip of precious metal 470 is deposited directly onto the first electrically conductive material layer 460, but this deposition is provided only in the first conductive region 440 located adjacent the distal end 410 of the test strip 400. According to this embodiment, a metallized layer of gold (not shown) is deposited onto the first conductive region of the upper base member 404 and a metallized layer of palladium 470 is deposited onto the first conductive region 440 of the lower base member 408. It will be understood that any precious metal can alternatively be selected for deposition including those from the group consisting of platinum, iridium, rhenium, ruthenium, rhodium and osmium. The remainder of each first electrically conductive material layer 460, is masked prior to depositing the precious metal layers 470, the latter which can be deposited using any suitable deposition technique, but preferably using either physical vapor deposition (PVD) or chemical vapor deposition (CVD) processes. In addition, this specific embodiment describes the use of two different precious metals (gold and palladium) on the cofacing electrodes, but it will be understood that according to at least one other version, the same precious metal can be deposited on both of the defined electrodes. At the time of manufacture, the sequential deposition of the film layers could occur within the same material run, or could occur in separate runs.

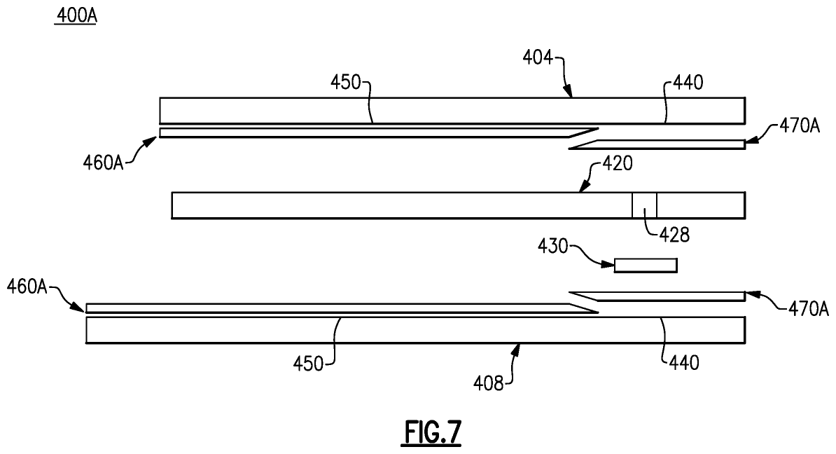

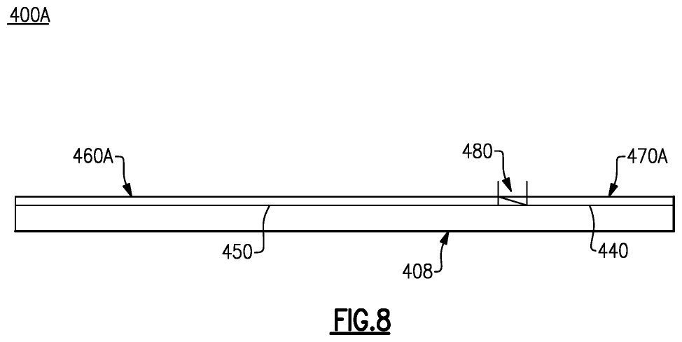

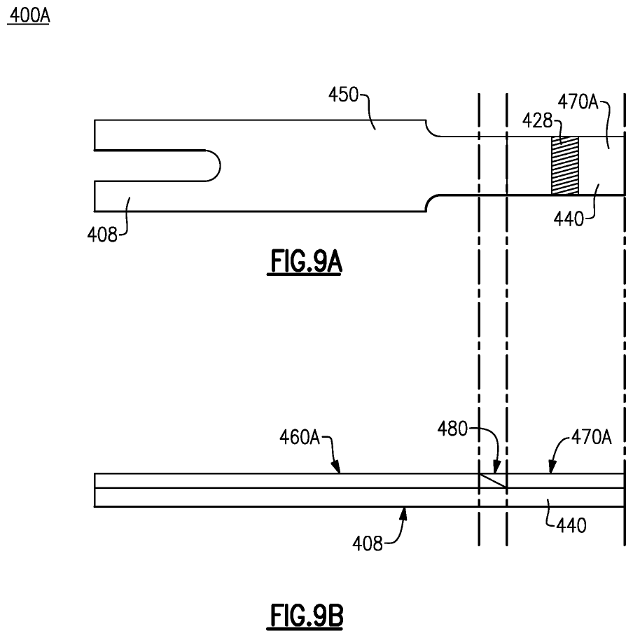

According to another embodiment and with reference to FIGS. 7-9(b), another test strip 400A is provided. Similar part numbers are identified herein using the same reference numbers for the sake of clarity. As in the preceding, the test strip 400A includes an upper base member 404, a lower base member 408, a spacer layer 420 having a cut out area 428 adjacent the distal end 410 of the test strip 400A and a reagent layer 430, which is disposed in the cut out area 428 directly above the lower base member 408. According to this embodiment, a first electrically conductive material layer 460A is deposited onto the facing surface of the upper and lower base members 104, 108 with the exception of the first conductive region 440, which is masked. The first electrically conductive layer 460A (i.e., non-precious metal) can include nickel, chromium, copper and silver and/or alloys, such as nichrome, may be used as the material for electrical signal transmission. As previously discussed, literally any material with an appropriate electrical conductivity may be used for the first electrically conductive material layer 460A. The first electrically conductive material layer 460A can be applied by any suitable deposition process over the entire interior facing surface of each of the upper and lower base members 404, 408 according to this specific embodiment. The first conductive region 440 at the distal end 410 of the test strip 400A is masked during the deposition of the first electrically conductive material (non-precious metal) layer 460A. A precious metal layer 470A is then deposited directly onto each base member 404, 408 and specifically onto the first conductive region 440 of each of the upper and lower base members 404, 408, covering the area which would become the working area of the test strip 400A. In this specific embodiment, gold or palladium is selected as the precious metal for the facing electrodes but it will understood that other precious metals, including those previously noted, can be utilized. According to this embodiment, there would be an overlap, which is schematically shown by reference number 480, of the first and second conductive material layers 460A, 470A extending over a distance that is compatible with the tolerance of the sputtering process, as best shown in FIGS. 8, 9(a) and 9(b).

Where differences in resistances exist (for example when using an alloy with a higher resistance than that of a precious metal) sputtered layer thicknesses may be increased or decreased for either the precious metal or the non-precious metal layer to alter and match electrical conductivity between the two layers to ensure that conductivity of the non-precious metal or alloy layer is not signal limiting. For example the thickness of the precious metal layer 470 of FIGS. 6(a) and 6(b) could be as thin as 5 nanometers, whereas for the seamed adjacent version of FIGS. 7-9(b), the thickness of the precious metal layer 470A would be thicker (e.g., at least 8 nanometers) due to the lack of an underlying metal layer.

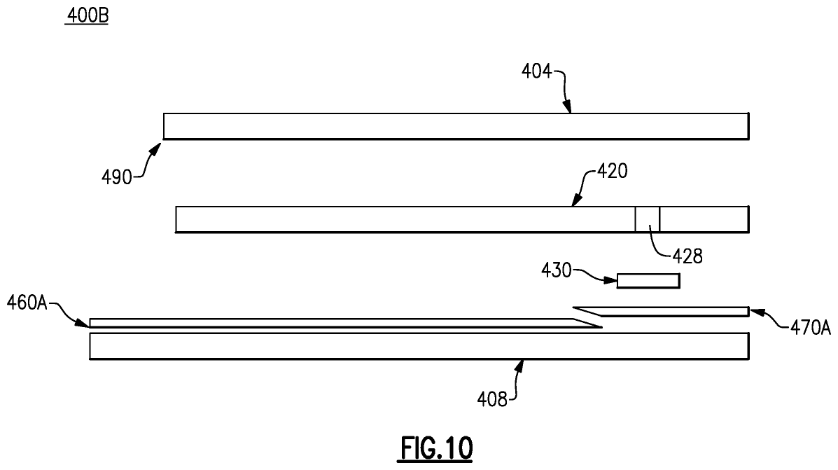

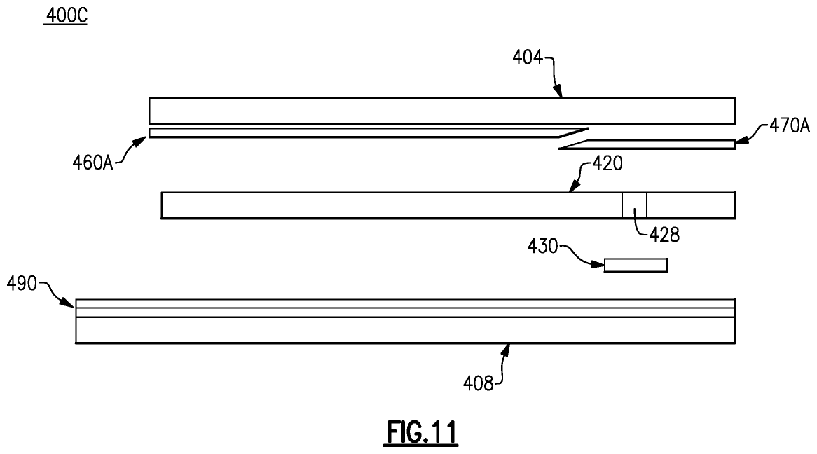

It should be understood that other variations and modifications are possible. With reference to FIGS. 10 and 11, there are shown test strips 400B and 400C in which a continuous precious metal layer 490 is placed over the entirety of the facing surface of one of the base members 404, FIG. 10 or 408, FIG. 11, with the “seamed” version employing the first and second conductive material layers 460A, 470A are applied to the other base member 408, 404. It will be understood that other variants and modifications can be made in accordance with aspects of the present invention.

In each of the embodiments described herein, the locally applied or deposited precious metal layers 470, 470A, for each test strip define respective cofacial electrodes that are separated by the cut out area 428 of the spacer layer 420 at the working area of the test strip. The overlapping portions of the non-precious material layers 460, 460A define electrode connection tracks that extend to contact pads provided at the opposing (proximal) end 414 of the test strip, thereby enabling connection to a test meter as previously discussed at FIGS. 2 and 3 for application of a suitable test voltage in the manner previously described.

### PARTS LIST FOR FIGS. 1-11

60 spacer layer
61 sample receiving chamber
62 test strip
63 contact pad
64 upper base member
65 U-shaped notch
66 lower base member
67 contact pads
67a prong
67b prong
68 cutout area
70 port(s)
72 reagent layer
76 electrode track
78 electrode track
80 distal end, test strip
82 proximal end, test strip
100 test meter
101 second electrode connector
102a first electrode connector
102b first electrode connector
106 test voltage unit
107 current measurement unit
164 first electrode
166 second electrode
202 display
210 memory unit
212 processor
400 test strip
400A test strip
400B test strip
400C test strip
404 upper base member
408 lower base member
410 distal end, test strip
414 proximal end, test strip
420 spacer layer
428 cut out area, spacer layer
430 reagent layer
440 first conductive region
450 second conductive region
460 first electrically conductive material layer
460A first electrically conductive material layer
470 second electrically conductive material layer
470A second electrically conductive material layer
480 overlap (seam)
490 continuous electrically conductive material layer
T₁ time interval
T₂ time interval
T₃ time interval
T_{G} glucose test time (sequence)

It will be understood that only a number of exemplary embodiments have been described herein and a number of variations and modifications embodying the inventive concepts will be readily apparent to a person of ordinary skill reading this application and in accordance with the following appended claims.

1. A biosensor comprising:
a first base member;
a second base member, the first and second base members being made from an insulative material wherein the first and second base members face on another in a spaced and parallel relationship;
at least one electrically conductive layer deposited onto a facing surface of each of the first and second base members, each facing surface including a first conductive region and a second conductive region adjacent the first conductive region wherein the first conductive region includes at least a first electrically conductive material and the second conductive region includes at least a second electrically conductive material different from the first electrically conductive material, wherein the first electrically conductive material is a precious metal and in which the first conductive regions are cofacial electrodes of the biosensor.

2. The biosensor according to claim 1, in which the precious metal is at least one of the group consisting of gold, palladium, platinum, osmium, rhodium, ruthenium, iridium, and rhenium.

3. The biosensor according to claim 1, in which the first conductive region of the first base member includes one of a first precious metal and the first conductive region of the second base member includes a second precious metal different than the first precious metal.

4. The biosensor according to claim 1, in which the first conductive region of the first and second base members includes the same precious metal.

5. The biosensor according to claim 1, in which the second electrically conductive material layer comprises at least one from the group including nickel, chromium, copper, silver, including alloys thereof.

6. The biosensor according to claim 1, in which the at least one electrically conductive layer includes a layer of the second electrically conductive material deposited on the first and second conductive regions of at least one of the first and second base members and a layer of the precious metal deposited directly onto the second electrically conductive material layer in the first conductive region.

7. The biosensor according to claim 1, in which the electrically conductive layer includes a layer of the first conductive material applied to the first conductive region and a layer of the second conductive material applied to the second conductive region.

8. The biosensor according to claim 6, in which the first conductive region and the second conductive region overlap one another.

9. The biosensor according to claim 7, in which the first conductive region and the second conductive region overlap one another.

10. The biosensor according to claim 1, further comprising a spacer layer disposed between the electrically conductive layers, the spacer layer including a spaced area between the first conductive regions of the first and second base layers, defining a functional area of the biosensor.

11. The biosensor according to claim 1, in which the second conductive region includes at least one contact pad configured for coupling to a test meter.

12. The biosensor according to claim 1, in which the precious metal layer is applied by a physical vapor deposition (PVD) or a chemical vapor deposition (CVD) process.

13. The biosensor according to claim 1, in which the second electrically conductive material is applied by a physical vapor deposition (PVD) or chemical vapor deposition (CVD) process.

14. A method for manufacturing a biosensor, the method comprising:
providing a first base member and a second base member;
depositing an electrically conductive layer onto surfaces of each of the first and second base members, each base member having a first conductive region and a second conductive region wherein a first electrically conductive material is deposited on a first conductive region of the base member and a second conductive material different from the first conductive material is deposited on a second conductive region of the base member adjacent the first conductive region in which the first electrically conductive material is a precious metal; and
disposing the first conductive regions of the first and second base members in spaced relation with one another and in which the first conductive regions form a pair of cofacial electrodes.

15. The method according to claim 14, further comprising:
providing a spacer layer between the electrically conductive layers, the spacer layer including a spacing formed between the first conductive regions.

16. The method according to claim 14, wherein the depositing an electrically conductive layer further comprises:
depositing a layer of the second electrically conductive material on the first and second conductive regions of the first and second base members; and then
depositing a layer of the first electrically conductive material onto the second conductive material layer in the first conductive regions.

17. The method according to claim 14, wherein the depositing an electrically conductive layer further comprises:
depositing a layer of the first electrically conductive material on the first conductive region of the first and second base members; and
depositing a layer of the second electrically conductive material on the second conductive region of the first and second base members.

18. The method according to claim 16, further comprising:
creating an overlap between the first and second electrically conductive materials between the first and second conductive regions.

19. The method according to claim 17, further comprising:
creating an overlap area between the first and second electrically conductive materials at the first and second conductive regions.

20. The method according to claim 14, wherein the first electrically conductive material is deposited by one of a physical vapor deposition (PVD) or chemical vapor deposition (CVD) process.

21. The method according to claim 14, wherein the second electrically conductive material is deposited by one of a physical vapor deposition (PVD) or chemical vapor deposition (CVD) process.

22. The method according to claim 14, further including forming electrical contact pads on the second conductive regions that are configured for engagement with a test meter.

23. The method according to claim 14, further comprising providing a reagent layer onto at least one of the first conductive regions and onto the precious metal.

24. The method according to claim 14, wherein the first conductive material deposited onto the first conductive region of one of the first and second base members is a first precious metal and the first conductive material deposited onto the first conductive region of the other of the first and second base members is a second precious metal, the second precious metal being different than the first precious metal.

25. The method according to claim 14, wherein the first conductive material deposited onto the first conductive regions of the first and second base members is the same precious metal.
